(19) United States
(12) Patent Application Publication
(10) Pub. No.: US 2009/0181981 A1
(43) Pub. Date: Jul. 16, 2009

(54) CRYSTALLINE (R)-2-(4-CYCLOPROPANESULPHONYL-PHENYL)-N-PYRAZIN-2-YL-3-(TETRAHYDROPYRAN-4-YL)-PROPIONAMIDE

(76) Inventors: Jeanette Tower Dunlap, Louisville, KY (US); Gregory Alan Stephenson, Fishers, IN (US)

Correspondence Address: ELI LILLY & COMPANY, PATENT DIVISION, P.O. BOX 6288, INDIANAPOLIS, IN, 46206-6288, US

(21) Appl. No.: 12/349,552

(22) Filed: Jan. 7, 2009

Related U.S. Application Data
(60) Provisional application No. 61/021,133, filed on Jan. 15, 2008.

(51) Int. Cl. A61K 31/497; C07D 405/12

(52) U.S. Cl. 514/255.05; 544/336

(57) ABSTRACT

Crystalline R-2-(4-cyclopropanesulfonyl-phenyl)-N-pyrazin-2-yl-3-(tetrahydropyran-4-yl)-propionamide and methods of its preparation and use are disclosed.

The present invention provides a crystalline form of R-2-(4-cyclopropanesulfonyl-phenyl)-N-pyrazin-2-yl-3-(tetrahydropyran-4-yl)-propionamide and a process for preparing the crystalline compound.

Diabetes is a growing at alarming proportions worldwide. The body's ability to produce sufficiently quantities and/or effectively use insulin to metabolize glucose results in a plethora of adverse conditions. Glucokinase is important in regulation of plasma glucose levels. It is thought that glucokinase activators (GK activators) increase a body's sensitivity to glucose. Consequently, GK activators are thought to be effective in the treatment of hyperglycemia, insulin resistance, and diabetes, particularly in type II diabetes.

The compound, R-2-(4-cyclopropanesulfonyl-phenyl)-N-pyrazin-2-yl-3-(tetrahydropyran-4-yl)-propionamide (herein after CPTP), illustrated below is useful as a Glucokinase (GK) activator as disclosed in WO2004/072031.

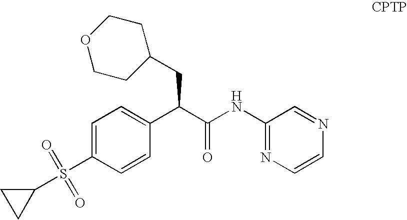

Amorphous CPTP can be prepared according to the procedures disclosed in that published PCT application and in WO2006/016178. However, the physical properties of the amorphous free base form make it highly undesirable for commercial processing and formulating into a pharmaceutically elegant drug product. The amorphous free base exhibits limited thermal stability and must be stored cold to inhibit degradation. Further the amorphous free base material is hygroscopic at ambient temperatures requiring storage in sealed containers to minimize adventitious introduction of moisture. Further the amorphous free base is not free a flowing powder, but is at best characterized as a “sticky” solid, which can entrap solvents and other impurities, and tends to clump together hindering processing and drug formulation.

These properties limit use of the amorphous material in solid, oral formulations. Similarly when a liquid formation is required, it may be necessary to dissolve or suspend the amorphous material in the pharmaceutically acceptable diluent just prior to dispensing or administration. These procedures add costs and limit the applicability or practicality of certain formulations.

Preparation of acid addition salts of CPTP is also challenging because of the relative low basicity of the amines in the compound. This limits the choice of suitable acids to the more acidic acids, which can cause decomposition of CPTP over time. This and other physical characteristics of CPTP have hampered attempts to prepare an isolatable, stable crystalline material.

A significant need exists for more effective treatment and control of hyperglycemia, insulin resistance, and diabetes. Further, there is a need to provide GK active compounds that are more stable, more readily purified, and more/or easily formulated into drug products. The present invention addresses these needs and provides related advantages as well.

In one form, the present invention provides R-2-(4-cyclopropanesulphonyl-phenyl)-N-pyrazin-2-yl-3-(tetrahydropyran-4-yl)-propionamide in crystalline form characterized by an X-ray powder diffraction pattern obtained from a CuKα source (λ=1.54056 Å), which comprises peaks at a) 11.5° and 19.0°±0.1° in 2θ; or b) 11.5°, 17.1°, 19.0°, and 26.6°±0.1° in 2θ; or c) 11.5°, 17.1°, 19.0°, 26.6°, 9.4°, 15.0°±0.1°, 16.5°±0.1°, and 20.7°±0.1° in 2θ.

In one form, the present invention provides R-2-(4-cyclopropanesulphonyl-phenyl)-N-pyrazin-2-yl-3-(tetrahydropyran-4-yl)-propionamide in crystalline form characterized by an X-ray powder diffraction pattern obtained from a CuKα source (λ=1.54056 Å), which comprises peaks at 11.5° and 19.0°±0.1° in 2θ. In another form, the present invention provides R-2-(4-cyclopropanesulphonyl-phenyl)-N-pyrazin-2-yl-3-(tetrahydropyran-4-yl)-propionamide, which further comprises peaks at 17.1° and 26.6°±0.1° in 2θ, and/or which further comprises peaks at 29.4°, 15.0, 16.5°, and 20.7°±0.1° in2θ.

In one form, the present invention provides R-2-(4-cyclopropanesulphonyl-phenyl)-N-pyrazin-2-yl-3-(tetrahydropyran-4-yl)-propionamide in crystalline form characterized by a solid state NMR pattern, which comprises peaks, relative to adamantane (δ=29.5 ppm) at δ 172.8, 49.7, and 43.5+/10.1 ppm. In another form, the present invention provides R-2-(4-cyclopropanesulphonyl-phenyl)-N-pyrazin-2-yl-3-(tetrahydropyran-4-yl)-propionamide, which further comprises peaks at δ 144.7 and 140.2±0.1 ppm, and/or δ 6.5, and 5.6±0.1 ppm.

In one form, the present invention provides R-2-(4-cyclopropanesulphonyl-phenyl)-N-pyrazin-2-yl-3-(tetrahydropyran-4-yl)-propionamide in crystalline form characterized by a solid state NMR pattern, which comprises peaks, relative to adamantane (δ=29.5 ppm) at δ 172.8, 49.7, and 43.5±10.1 ppm. In another form the present invention provides R-2-(4-cyclopropanesulphonyl-phenyl)-N-pyrazin-2-yl-3-(tetrahydropyran-4-yl)-propionamide in crystalline form characterized by a solid state NMR pattern, which further comprises peaks, relative to adamantane (δ=29.5 ppm) at δ 144.7 and 140.2±0.1 ppm and/or at δ 6.5 and 5.6±0.1 ppm.

In another form, the present invention provides R-2-(4-cyclopropanesulphonyl-phenyl)-N-pyrazin-2-yl-3-(tetrahydropyran-4-yl)-propionamide in crystalline form characterized by at least one of the following: a) an X-ray powder diffraction pattern which comprises intense peaks at 11.5° and 19.0° in 2θ obtained from a CuKα source (λ=1.54056 Å); or b) an onset of melting using differential scanning calorimetry of 156±3° C.; or c) a solid state NMR patter which comprises peaks at 172.8, 49.7, and 43.5 ppm referenced to adamantine (δ=29.5 ppm).

In another form, the present invention provides R-2-(4-cyclopropanesulphonyl-phenyl)-N-pyrazin-2-yl-3-(tetrahydropyran-4-yl)-propionamide comprising substantially pure crystalline R-2-(4-cyclopropanesulphonyl-phenyl)-N-pyrazin-2-yl-3-(tetrahydropyran-4-yl)-propionamide.

In another form, the present invention provides a pharmaceutical composition comprising crystalline R-2-(4-cyclopropanesulphonyl-phenyl)-N-pyrazin-2-yl-3-(tetrahydropyran-4-yl)-propionamide and at least one other anti-diabetic agent or anti-hyperglycemic agent.

In another form, the present invention provides a pharmaceutical composition that includes R-2-(4-cyclopropanesulfonyl-phenyl)-N-pyrazin-2-yl-3-(tetrahydropyran-4-yl)-propionamide according to the present invention in a pharmaceutically acceptable carrier, diluent or excipient.

In yet another form, the present invention provides the use of R-2-(4-cyclopropanesulphonyl-phenyl)-N-pyrazin-2-yl-3-(tetrahydropyran-4-yl)-propionamide according to the present invention for the manufacture of a medicament for the prevention of hyperglycemia.

In another form, the present invention provides for the use of a R-2-(4-cyclopropanesulphonyl-phenyl)-N-pyrazin-2-yl-3-(tetrahydropyran-4-yl)-propionamide according to the present invention for the manufacture of a medicament for the treatment of diabetes or hyperglycemia.

In another form, the present invention provides R-2-(4-cyclopropanesulphonyl-phenyl)-N-pyrazin-2-yl-3-(tetrahydropyran-4-yl)-propionamide according to the present invention for use in therapy.

In another form, the present invention provides R-2-(4-cyclopropanesulphonyl-phenyl)-N-pyrazin-2-yl-3-(tetrahydropyran-4-yl)-propionamide according to the present invention for use in the treatment of diabetes or hyperglycemia.

In still yet another form, the present invention provides a method of preventing or treating hyperglycemia in a mammal including humans in need of treatment. The method comprises administering an effective amount of R-2-(4-cyclopropanesulphonyl-phenyl)-N-pyrazin-2-yl-3-(tetrahydropyran-4-yl)-propionamide according to the present invention to a patient in need of treatment. The patient includes human and non-human mammals in need of treatment.

In another form, the present invention provides method of treating diabetes in a mammal, including humans in need of treatment. The method comprises administering an effective amount of R-2-(4-cyclopropanesulphonyl-phenyl)-N-pyrazin-2-yl-3-(tetrahydropyran-4-yl)-propionamide according to the present invention. Optionally the method can also include administration of at least one other anti-diabetic agent or anti-hyperglycemic agent.

Crystalline R-2-(4-cyclopropanesulphonyl-phenyl)-N-pyrazin-2-yl-3-(tetrahydropyran-4-yl)-propionamide can be prepared by dissolving the amorphous CPTP material (or generating the CPTP material in situ) in a polar solvent such as, but not restricted to, isopropyl alcohol, ethanol, ethyl acetate and either cooling the resulting solution/mixture or adding an anti-solvent, such as, hexane, cyclohexane, or heptane and the like.

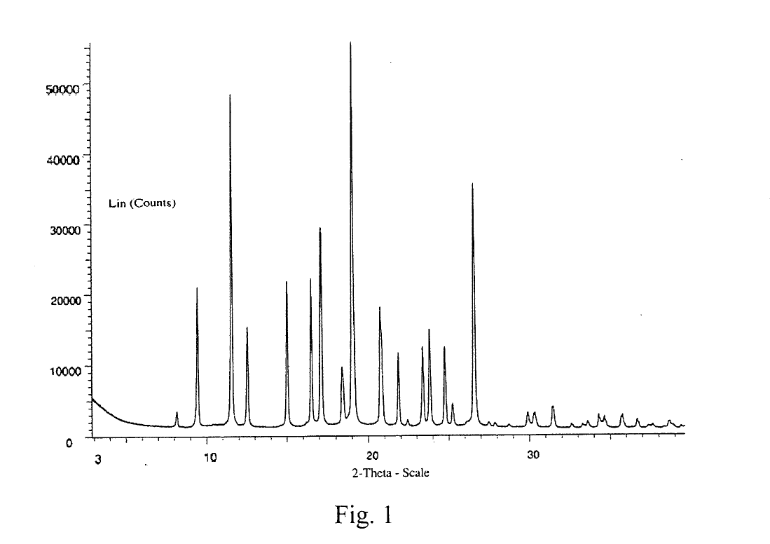

FIG. 1 is a spectrogram of a representative XRD pattern for crystalline CPTP. The XRD spectrogram was obtained as described in the Experimental Section below.

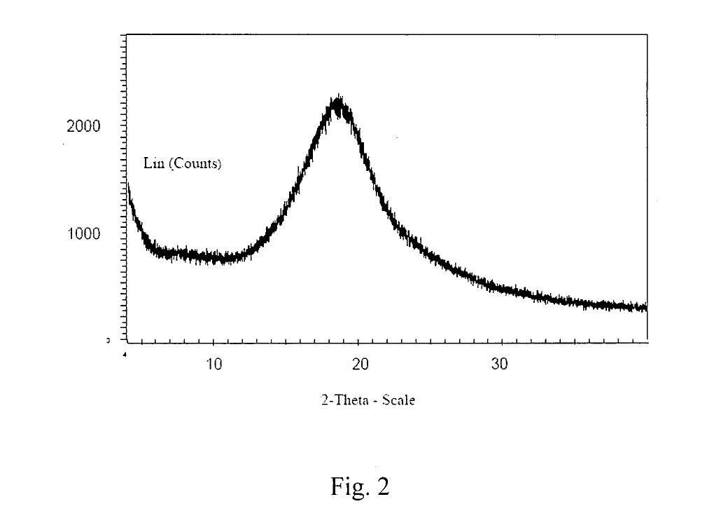

FIG. 2 is a spectrogram of a representative XRD pattern for amorphous CPTP. The XRD spectrogram was obtained according to the procedure described for crystalline CPTP in the Experimental Section below.

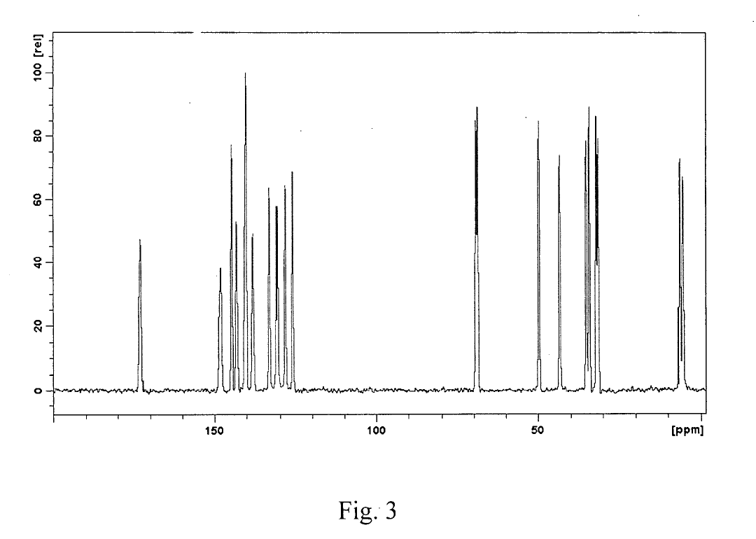

FIG. 3 is a spectrogram of a representative solid state NMR pattern for crystalline CPTP. The solid state NMR spectrogram was obtained according to the procedure described in the Experimental Section below.

Glucokinase is important in regulation of plasma glucose levels. It is thought that GK activators increase a body's sensitivity to glucose. Consequently, GK activators are effective in the treatment of hyperglycemia, insulin resistance, and diabetes, particularly in type II diabetes. Amorphous CPTP has been demonstrated to be active in both an in-vitro and in-vivo GK assays. (WO2004/072031) However, amorphous CPTP is not suitable for pharmaceutically elegant drug formulation. The amorphous material exhibits limited thermal stability as evidenced by its low glass transition temperature (Tg (° C.): 68 to 77° C. affected by thermal and solvent history); is hygroscopic; and does not provide a free flowing powder.

Attempts to find more stable and suitable forms have been extensively investigated. Table 1 below provides a representative listing of attempts to crystallize amorphous CPTP from a variety of different solvents including ethanol, methyl t-butyl ether, acetone, isopropanol, ethyl acetate, either with or without an anti-solvent, such as, heptane, toluene, and water. As noted in the Table 1 these attempts failed to provide crystalline CPTP.

TABLE 1
Attempts to Crystallize Amorphous CPTP (98% HPLC purity)

| Solvent | | | Temp. | |
|---|---|---|---|---|
| A | B | Ratio A:B | (° C.) | Remarks |
| t-Butylmethyl | Heptane | 1:9 | RT | white amorphous solid |
| ether | | | | |
| Ethanol | Heptane | 1:9 | RT | oily residue forms |
| Acetone | Heptane | 1:9 | RT | oily residue forms |
| i-Propanol | Heptane | 1:19 | RT | oily residue forms |
| Ethanol | Toluene | 1:9 | RT | oily residue forms |
| i-Propyl acetate | Heptane | 1:9 | RT | suspension and then an |
| | | | | oily residue forms |
| | | | | after 1 h stirring |
| i-Propyl acetate | Heptane | 0.5:9.5 | RT | White amorphous solid |
| Ethyl acetate | Heptane | 1:9 | RT | oily residue forms |
| t-Butylmethyl | H₂O | A saturated | RT | oily residue forms |
| ether | | with B | | |
| t-Butylmethyl | Heptane | 1:9 | 40 | white amorphous solid |
| ether | | | | |
| t-Butylmethyl | Heptane | 1:9 | 60 | oily residue forms |
| ether | | | | |

Salt formation was also extensively investigated. However, formation of isolable CPTP salts is hampered by the low pK (pKₐ₁=0.17, determined by photometric titration) of CPTP. Table 2 below provides a representative listing of attempts to form crystalline CPTP salts.

TABLE 2
Salt Screen

| | CPTP | | Anti- | |
|---|---|---|---|---|
| Acid | (g) | Solvent | solvent | Remarks |
| p-Tolunesulfonic | 1 | Ethanol | Diethyl | Yellow oily residue after |
| acid | | | ether | 30 min stirring |
| HCl gas | 2 | Dioxane | Diethyl | Oil which solidified after |
| | | | ether | trituration |
| 4 M HCl/Dioxane | 0.5 | Dioxane | Diethyl | Oily Residue |
| | | | ether | |
| 6 M HCl/ | 0.5 | Dioxane/ | Diethyl | Sticky suspension, very |
| i-Propanol | | i- | ether | hygroscopic in air |
| | | Propanol | | |
| H₂SO₄, 96% tech. | 1 | Ethanol | Diethyl | Yellow oily residue |
| | | | ether | |

Unexpectedly it has now been discovered that crystalline CPTP can be prepared. Originally crystalline CPTP was discovered during salt screening attempts to prepare the base addition salt (instead of the acid addition salt) after forming the CPTP anion by adding a variety of different bases, for example, sodium hydroxide, potassium hydroxide, and diethyl amine among other bases. While the base addition salt was not identified, a white crystalline material did precipitate out of the reaction medium. The white crystalline material was identified as crystalline CPTP (as the free base). The procedure later repeated on a larger scale again yielded the same crystalline CPTP.

Crystalline CPTP can be prepared from amorphous CPTP under suitable conditions with or without seed crystals. Typically, CPTP can be crystallized from a single or mixed solvent system prepared by first dissolving amorphous CPTP in polar solvent then adding an anti-solvent (or non-polar solvent). Alternative procedures can also employ repeated heating and cooling cycles to modify crystalline particle size. Examples of polar solvents for use in this invention include, but are not restricted to, ethyl acetate; ketones, such as, acetone or methyl ethyl ketone (MEK); ethers, such as methyl tert-butyl ether (MTBE). Alcohols, for example, ethanol and isopropyl alcohol, can also be used. Typical non-polar solvents for use in this invention include alkanes and cycloalkanes such as n-heptane or cycloheptane. Mixtures of the alkanes, for example, mixtures of hexanes or heptanes, although less preferred, can also be employed. Preferred solvents include isopropyl alcohol and ethyl acetate either with or without an anti-solvent. Additionally, it is preferred that the solvent system be anhydrous.

A typical crystallization process can include heating suspended CPTP in a single or mixed solvent system to between about 50° C. and about 70° C., with or without stirring or sonication to affect dissolution, then cooling the resulting solution to a temperature level between ambient temperature and about 0° C. with stirring for a period of time to allow crystals to form. If crystals do not form, then a seed crystal can be added. If desired, the solution can be subjected to repeated heating/cooling cycles to modify crystal particle size.

In one preferred procedure, it has been found that impurities and/or moisture found in amorphous CPTP inhibit crystallization. The impurities can be removed by acid/base extractions, chromatography, or precipitation, repeatedly if necessary, from a mixed solvent system prior to crystallization.

In one procedure, crystalline CPTP can be prepared by removing the solvent used in the purification procedures for the synthesis of amorphous CPTP; re-dissolving the amorphous CPTP in warm isopropyl alcohol; and then cooling the resulting solution to effect crystallization of CPTP. A seed crystal of previously purified CPTP can be added to the cooled solution to facilitate the crystallization process.

The above crystallization procedure provides substantially pure crystalline CPTP. As used herein the term “substantially pure” refers to a composition comprising greater than 80% w/w of the crystalline CPTP, preferable greater than 95% w/w, and yet more preferable greater than 98% w/w of crystalline CPTP.

The crystalline CPTP exhibits superior properties over those of amorphous CPTP. The superior properties include, inter alia, better thermal, chemical stability, and processability. Crystalline CPTP can be stored at ambient temperature with minor or no degradation. Crystalline CPTP has an onset of melting as measured by differential scanning calorimetry of 156° C., which renders it acceptable for standard industrial processes such as milling. Further, crystalline CPTP remains anhydrous and is not hygroscopic when stored at ambient temperature. Table 3 below lists the stability data for crystalline CPTP material.

TABLE 3
Crystalline CPTP Stability Summary

| | 25° C. 60% | | | 40° C. 60% | | |
|---|---|---|---|---|---|---|
| | relative humidity | | | relative humidity | | |
| | | | 3 | | 1 | 3 |
| | Initial | 1 month | months | Initial | Month | Months |
| Appearance | White | White | White | White | White | White |
| | Powder | Powder | Powder | Powder | Powder | Powder |
| Water (% w/w) | <0.07 | <0.10 | <0.01 | <0.07 | <0.10 | <0.01 |
| Assay (% w/w) | 100.3 | 99.9 | 99.6 | 100.3 | 100.1 | 99.3 |
| Chiral Purity† | 100 | 100 | 100 | 100 | 100 | 100 |
| (% a/a) | | | | | | |
| Achiral Purity‡ | 0.25 | 0.18 | 0.23 | 0.25 | 0.20 | 0.25 |
| (% a/a) | | | | | | |
| Total | 0.25 | 0.18 | 0.23 | 0.25 | 0.20 | 0.25 |
| Impurities | | | | | | |

†As measured by area percent using chiral HPLC analyis.
‡Total impurities measured are percent using achiral HPLC analysis.

Crystalline CPTP is a free flowing powder suitable for formulating into a drug product or pharmaceutical composition. It can be readily formulated into pharmaceutical compositions such as tablets, solid or gel filled capsules, powders, suspensions, or solutions. The pharmaceutical composition can comprise crystalline CPTP in amounts between 1% and 75% w/w, and more preferable 10 to 65% w/w. The composition can also include one or more pharmaceutically acceptable carriers, excipients and diluents. Non limiting examples of pharmaceutically acceptable carriers, excipients, and diluents are suitable for such formulations include the following: starch, sugars, mannitol, and silica derivatives; binding agents such as carboxymethyl cellulose and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium, and magnesium stearate, and solid polyethyl glycols.

Preferred pharmaceutical compositions include crystalline CPTP formulated as a tablet or capsule for oral administration. The tablet or capsule can include crystalline CPTP in amount between about 4 mg to about 300 mg, more preferably between about 40 mg and about 260 mg per tablet. In one embodiment, the table or capsule can be formulated to provide a sustained release of CPTP to the patient allowing a single or twice a day dosing regime.

The pharmaceutical composition is administered to a patient in amounts effective to treat or prevent hyperglycemia, insulin resistance or diabetes. An appropriate amount or dose effective to treat a patient can be determined by a health care provider. In one form the pharmaceutical composition can be administered in an amount sufficient to provide a patient with between 1 and 20 mg/kg patient/day and more preferably between about 2.5 to 15 mg/kg patient/day of CPTP.

The crystalline compound and compositions of the present invention may be employed in combination with one or more other anti-diabetic agents or anti-hyperglycemic agents. Examples of these agents include, sulfonylureas (e.g. glyburide, glimepiride, glipyride, glipizide, chlorpropamide, gliclazide, glisoxepid, acetohexamide, glibornuride, tolbutamide, tolazamide, carbutamide, gliquidone, glyhexamide, phenbutamide, tolcyclamide, etc.), biguanides (e.g. metformin, phenformin, buformin, etc.), glucagon antagonists (e.g. a peptide or non-peptide glucagon antagonist), glucosidase inhibitors (e.g. acarbose, miglitol, etc.), insulin secetagogues, insulin sensitizers (e.g. troglitazone, rosiglitazone, pioglitazone, etc.) and the like; or anti-obesity agents (e.g. sibutramine, orlistat, etc.) and the like. The compound and compositions of the present invention and the other anti-diabetic agents or anti-hyperglycemic agents may be administered simultaneously in a single delivery form, i.e. a single table, capsule or solution; in separate delivery forms administered simultaneously, sequentially, or at separate time periods.

The compound CPTP can be prepared according to the procedure illustrated below in Scheme 1 and more specifically described in the following preparations and Examples.

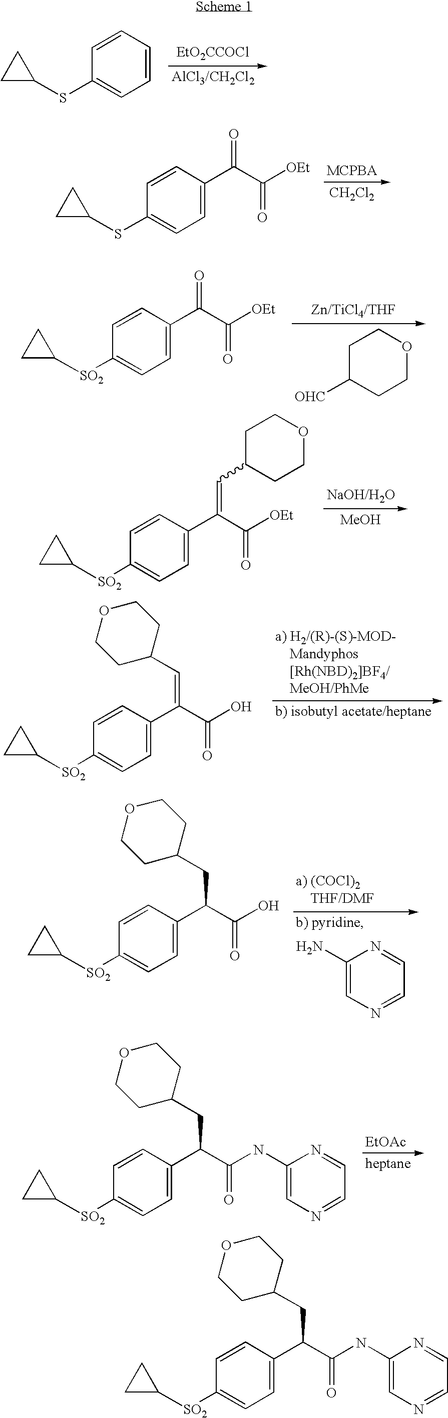

### Preparation 1. Ethyl 4-(cyclopropylsulfonyl)phenylglyoxylate

Charge a 2-L, three-necked, round bottomed flask equipped with a mechanical stirrer, thermometer, calcium chloride drying tube, and a pressure equalizing addition funnel with a mixture of dichloromethane (1490 mL) and aluminum chloride (190.0 g, 1.420 mol); cool the resulting mixture to −5° C. using a salt-ice bath. Dropwise add ethyl chlorooxoacetate (124.3 mL, 1.420 mol) as a yellow suspension over 1 hour at −5° C. Stir the resulting brown solution for 30 min at 0° C. Remove the cooling bath, and add cyclopropyl phenyl sulfide (148.3 mL, 1.030 mol) over 2 hrs at 18-20° C. (The brown solution turns a violet color after the addition of 4-5 drops of cyclopropyl phenyl sulfide, and in the last 10 min of addition, gas evolves from the reaction mixture.) Stir the reaction mixture for 4 hrs at 20° C. The reaction mixture can be monitored for completion by GC and TLC (eluent:n-hexane:ethyl acetate=6:4). Add the resulting reaction mixture to deionized water (2 L) at 10-15° C. with ice cooling. Separate the organic phase, and extract the aqueous phase with dichloromethane (350 mL). Combine the organic layers, and wash the combined organic layers with 500 mL of saturated NaHCO₃. Remove the dichloromethane under reduced pressure to provide 231.5 g (90% yield) of the title compound as a dark brown oil.

### Preparation 2. Ethyl 4-(cyclopropylsulfonyl)phenylglycoxylate

Charge a 2-L, three-necked round bottom flask equipped with a mechanical stirrer, addition funnel, and thermometer, with a mixture of ethyl 4-(cyclopropylthio)phenylglyoxilate (80.0 g, 320 mmol) and dichloromethane (640 mL). Cool the solution to +5° C., and add a solution of MCPBA (meta chloro perbenzoic acid) (144.0 g, 640 mmol) in dichloromethane (620 mL) at +5 to +20° C. over 1 hr. Stir the resulting reaction mixture for 1 hr at room temperature. Completion of the reaction can be monitored by GC and TLC (eluent:n-hexane:ethyl acetate=6:4). After the reaction is deemed complete, add 1 L of 2M NaHCO₃ over 20 min at 20° C. during which time gas evolution can be observed. Separate the organic and aqueous layers. Extract the aqueous layer with 100 mL of dichloromethane. Combine the organic layers, and sequentially wash the combined organic layers with 300 and 200 mL of water. Evaporate the dichloromethane under reduced pressure to provide 88.1 g (97.5% yield, 95% purity via GC) of the title compound.

### Preparation 3. Ethyl 2-(4-cyclopropanesulfonylphenyl)-3-(tetrahydropyran-4-yl)acrylate

Ethyl 2-(4-cyclopropanesulfonylphenyl)-3-(tetrahydropyran-4-yl)acrylate can be prepared as described in U.S. Pat. No. 7,214,681, Preparation 23.

### Preparation 4. (E)-2-(4-Cyclopropanesulfonylphenyl)-3-(tetrahydropyran-4-yl)acrylic acid

Charge a 1600 liter reactor with 200±5 kg of deionized water, 16.5±2 kg of sodium hydroxide and agitate the resulting mixture to dissolve the solids. After dissolution add 255±5 kg (330 L) methanol and 55±2 kg ethyl 2-(4-cyclopropanesulfonylphenyl)-3-(tetrahydropyran-4-yl)acrylate. Warm the resulting mixture to 65-70° C. and agitate the mixture for 1 hr while maintaining the temperature level between 65-70° C. Thereafter cool the mixture to 30° C. and sample the reaction to determine the amount of starting material, ethyl 2-(4-cyclopropanesulfonylphenyl)-3-(tetrahydropyran-4-yl)acrylate, still present (HPLC). Continue agitating the mixture until the starting material is less than 1% (area percent measured via HPLC). Remove the solvents in vacuo (0.8-0.9 bar) at 30-35° C. (330-350 L distillate). Then add 350±10 kg of deionized water and agitate for 1 hr. Remove the resulting solid triphenyl phosphine bi-product via filtration and/or via a centrifuge. Sequentially wash the solid with 1.5±0.1 kg sodium hydroxide dissolved in 50 ±1 kg deionized water and then with 30±1 kg deionized water. Collect the aqueous washings and extract with dichloromethane (2×75 L). Acidify the aqueous layer with 46±1 kg hydrochloric acid diluted with 370±10 kg deionized water (pH=1). Extract the acidified aqueous layer with methyl tert butyl ether (MTBE) (1× with 360 L, then 2× with 170 L). Combine the MTBE extracts and wash with deionized water (90±5 kg). Warm the resulting MTBE solution to 40-45° C. and add activated charcoal (1.5±0.2 kg). After about 1 hr filter off the charcoal and wash the charcoal with 25±1 kg MTBE. Remove the MTBE solvent in vacuo (0.8-0.9 bar) to a volume of approximately 80 L. Crystallize the title compound from the solvent at 0-5° C. Recrystallize the compound from methanol and dry the crystallized compound in vacuo without heating to yield between 15-20 kg of the titled compound.

### Preparation 5. (R)-2-(4-Cyclopropanesulfonylphenyl)-3-(tetrahydropyran-4-yl)propionic acid

Charge a 50 L steel autoclave with 2.9 kg of (E)-2-(4-cyclopropanesulfonyl phenyl)-3-(tetrahydropyran-4-yl)acrylic acid dissolved in 23.8 L of a solvent mixture of methanol/toluene (5/1). Close and then inertize the autoclave (pressurize 10× to 10 bar with N₂. Stir the solution for 1 min at 800 rpm. Charge a separate 0.5 L glass vessel.0.2 L with the solvent mixture methanol/toluene 5/1. Inertize the glass vessel by applying three consecutive argon/vacuum cycles. Thereafter add to the inertized glass vessel 6.14 g [Rh(NBD)₂]BF₄ (17.2 mmol, 0.21 mol %, s/c=500, where NBD is norbornadiene) and 18.2 g of (αR,αR)-1,1′-bis[α-(dimethylamino)benzyl]-(S,S)-,2′-bis {bis[3,5-bis(trifluoromethyl)phenyl]phosphino}ferrocene ((R)-(S)-MOD-Mandyphos, 18.1 mmol, 0.21 mol %) under argon. (See WO 06/161178) Inertize the glass vessel one more time by applying three consecutive argon/vacuum cycles. Stir the solution for 30 min under argon. Transfer the contents of the glass vessel via a cannula wire under nitrogen into the autoclave. Close and inertize the autoclave with N₂ (10 bar) and once with H₂ (10 bar). Set the pressure in the autoclave to 50 bar and check for leaks. Thereafter conduct the hydrogenation under H₂ 50 bar, with stirring (1000 rpm) for 20 h at 30° C. Thereafter stop stirring, release the pressure in the autoclave and sample the reaction mixture to ensure completion of the reaction. Unload the reaction solution from the autoclave into a 100 L vessel and add 1.4 kg (50 w/w %) of the adsorber MSA FC C-1 (formerly W-HP-04-69P) from Engelhard. Stir the resulting suspension for 2 h at room temperature. Filter the solution through a pad of ca 500 g CELITE. Collect the filtrate, and wash the CELITE pad twice with 4 L of methanol. Combine methanol washing with the filtrate. Remove the solvent from the combined filtrate and methanol washings under reduced pressure to provide the title compound (2,299 g, 89% yield, 86% ee). Alternative catalyst adsorbers, in place of MSA FC Cl, can also be used including Monmorrillonit K10 or Degussa Charcoal 109.

### Preparation 6. Preparation of CPTP

Charge a 10 L reaction vessel, purged with N₂ with dichloromethane (2 L) and DMF (54.7 mL, 0.709 mol) and cool to −10° C. Slowly add oxalylchloride (60.0 mL, 0.709 mol) over 15 min. Strong gas evolution can be observed and a white suspension forms. Continue stirring the mixture until no further gas evolution is observed, then cool the resulting suspension to −20° C. Add a suspension of the acrylic acid (200 g 0.590 mol) in dichloromethane 1 L) over a 1 h period. Stir the resulting yellow solution for an additional 0.5 h, then cool to −45° C. Add to the cooled solution, pyridine (51.0 mL, 0.590 mol) in dichloromethane (0.25 L) over a 20 min. While maintaining the temperature under −45° C., add a suspension of 2-aminopyrazine (112.4 g, 1.180 mol) in THF (1.1 L) and a solution of pyridine (154 mL, 1.770 mol) in dichloromethane (0.9 L) in parallel over 1.25 h. Thereafter, remove the cooling bath and stir the resulting orange suspension for an additional 16 h. Concentrate the solution under reduced pressure and add ethyl acetate in 3×800 mL portions, the remove the solvents under reduced pressure to provide a brown oil. Add ethyl acetate (2 L) and aqueous HCl (2 M, 2 L). Separate the organic and aqueous phases, and extract the organic phase with ethyl acetate (2 L). Collect the organic phases and sequentially wash the combined organic phases with water (2 L) and a saturated aqueous of NaHSO₃ (2×1 L). The aqueous phase can be analysis via HPLC to determine if it contains additional product. If so an additional amount of the titled compound can be obtained by extracting the aqueous phase again with additional ethyl acetate (2 L). Collect all the organic factions, wash the collected fractions with water (1 L), and brine (1 L), dry the resulting fractions with Na₂SO₄ and filter. Collect the filtrate and remove the solvent under reduced pressure add ethanol portionwise (3×0.5 L) removing the organic solvents each time under reduced pressure to yield the title compound as brown oil (248 g, 96.5% pure via HPLC and ¹H NMR, 84% corrected yield and >98.0% ee). FIG. 2 is a spectrogram of a representative XRD pattern for amorphous CPTP prepared as described in this preparation.

The reaction yield can be improved by rigorously keeping moisture from the reaction. One source of moisture can be the 2-aminopyrizine reagent. Purification and drying this reagent by recrystallization from toluene reduces its water content to near 0% w/w.

### Preparation 7. Alternative Preparation of CPTP

Charge a 10 L reaction vessel, purged with N₂ with dichloromethane (2 L) and DMF (54.7 mL, 0.709 mol) and cool to −10° C. Slowly add oxalylchloride (60.0 mL, 0.709 mol) over 15 min. Strong gas evolution can be observed and a white suspension forms. Continue stirring the mixture until no further gas evolution is observed, then cool the resulting suspension to −20° C. Add a suspension of the acrylic acid (200 g 0.590 mol) in dichloromethane 1 L) over a 1 h period. Stir the resulting yellow solution for an additional 0.5 h, then cool to −45° C. Add to the cooled solution, pyridine (51.0 mL, 0.590 mol) in dichloromethane (0.25 L) over a 20 min. While maintaining the temperature under −45° C., add a suspension of 2-aminopyrazine (112.4 g, 1.180 mol) in THF (1.1 L) and a solution of pyridine (154 mL, 1.770 mol) in dichloromethane (0.9 L) in parallel over 1.25 h. Thereafter, remove the cooling bath and stir the resulting orange suspension for an additional 16 h. Concentrate the solution under reduced pressure and add ethyl acetate in 3×800 mL portions, the remove the solvents under reduced pressure to provide a brown oil. Add ethyl acetate (2 L) and aqueous HCl (2 M, 2 L). Separate the organic and aqueous phases, and extract the organic phase with ethyl acetate (2 L). Collect the organic phases and sequentially wash the combined organic phases with water (2 L) and a saturated aqueous of NaHSO₃ (2×1 L). The aqueous phase can be analysis via HPLC to determine if it contains additional product. If so an additional amount of the titled compound can be obtained by extracting the aqueous phase again with additional ethyl acetate (2 L). Collect all the organic factions, wash the collected fractions with water (1 L), and brine (1 L), dry the resulting fractions with Na₂SO₄ and filter. Collect the filtrate and remove the solvent under reduced pressure add ethanol portionwise (3×0.5 L) removing the organic solvents each time under reduced pressure to yield the title compound as brown oil (248 g, 96.5% pure via HPLC and ¹H NMR, 84% corrected yield and >98.0% ee). FIG. 2 is a spectrogram of a representative XRD pattern for amorphous CPTP prepared as described in this preparation.

The reaction yield can be improved by rigorously keeping moisture from the reaction. One source of moisture can be the 2-aminopyrizine reagent. Purification and drying this reagent by recrystallization from toluene reduces its water content to near 0% w/w.

### EXAMPLE 1

### Initial Preparation of Crystalline Form of CPTP

Initially, crystalline CPTP was isolated by scaling up one of the promising hits from the salt screen, in which an attempt was made to prepare the lysine salt. A quantity (120.6 mg) of the CPTP was weighed into a vial and then 1 mL acetone was added to the vial The sample was heated to ˜50° C. with stirring. An equivalent molar amount of L-lysine was dissolved in minimal water and added to the CPTP solution. After a few hours, the sample was cooled to ˜25° C. The sample was evaporated under a stream of nitrogen resulting in an oil. Methyl ethyl ketone (MEK, ˜3 mL) was added to the oil with sonication and then stirred at ˜60° C. An oil remained. After ˜2 hours of no change, the sample was cooled to ˜25° C. and left uncapped at ambient temperature. Ethyl acetate (EtOAc, ˜3 mL) was added to the oil while stirring at ˜60° C. The sample was cooled to ˜25° C. The clear solution was evaporated under a stream of nitrogen resulting in an oil. Methyl tert-butyl ether (MTBE, ˜3 mL) was added to the oil with sonication and then stirred at ˜60° C. An oil remained. After ˜2 hours of no change, the sample was cooled to ˜25° C. and left uncapped at ambient temperature over night and a solid crystalline material resulted, which was characterized as crystalline free base CPTP by X-ray powder diffraction.

### EXAMPLE 2

### Preparation of Crystalline Form CPTP

Charge a vial with 529 mg of amorphous CPTP and add 3×200 uL ethanol (EtOH) aliqouts while shaking. White solids precipitate from the sample. There after add additional EtOH to provide a total volume of 2 mL. The white solids remain undissolved. Stir the resulting mixture at ambient temperature for several hours. Isolate the solids by vacuum filtration to recover 73% percent yield based on the amount of the original amorphous material. Inspection of the solid reveals needle shaped crystals. These crystals can be used as seed crystals for subsequent crystallization procedures.

### EXAMPLE 3

### Preparation of Crystalline Form CPTP from Ethyl Acetate and Hexane

Single crystals suitable for X-ray diffraction can be grown by dissolving approximately 25 mgs of amorphous CPTP in 2 mL of ethyl acetate and then diffusing hexane vapor at room temperature until large crystals appeared in the bottom of the vial.

### Example 4

### Preparation of Crystalline Form CPTP from Isopropyl Alcohol

Suspend the crude material from the amide coupling reaction according to Preparation 6 in 4 vol. of isopropyl alcohol and heat the mixture to 80° C. until the solids dissolve. Slowly cool the mixture to 20° C. to induce crystallization. Alternatively, suspend the crude material in 4 vol. of isopropyl alcohol and heat the suspension to 70° C. and slowly cool the resulting mixture to 20° C. to induce crystallization if CPTP.

### EXAMPLE 5

### Preparation of Crystalline Form CPTP from Isopropyl Alcohol with Thermal Cycling

Suspend the crude material from the amide coupling reaction according to Preparation 6 in 4 vol. of isopropyl alcohol and heat the mixture to 75° C. Slowly cool the mixture to 40° C. to induce crystallization. Re-heat the resulting suspension to 65° C. and cool slowly to 40° C. Again, re-heat the resulting suspension to 60° C. and cool slowly to 20° C. Collect crystalline CPTP.

### EXAMPLE 6

### CPTP

Charge a 250 mL round bottom flask equipped with an over head stirrer, thermocouple and a nitrogen inlet line, with (R)-2-(4-Cyclopropanesulfonylphenyl)-3-(tetrahydropyra-4-yl)propionic acid H N Benzyl-α-methylbenzylamine (10 g, 29.55 mmol), THF (100 mL), and DMF (100 μL) to provide a hazy yellow solution. Sweep nitrogen gas over the reaction mixture for several minutes. Thereafter add oxalyl chloride to the mixture while maintaining the temperature at about 20° C. Assay the reaction mixture after about 1 hr to determine if the formation of the acid chloride of CPTP is complete. In a second 250 round bottom flask equipped with an over head stirrer, thermocouple and nitrogen inlet line, add 2-aminopyrazine (3.09 g, 32.5 mmol), THF (50 mL), and pyridine (24 mL). Analyze this solution for water. The water content should preferably below 0.03%. Add 4 A molecular sieves if necessary to remove water from the solution. When the solution is sufficiently dry, add a 125 additional funnel to the round bottom flask and charge the funnel with the CPTP acid chloride solution. Drop-wise add the CPTP acid chloride solution to the second round bottom flask while maintaining the reaction mixture at 20° C. to yield a yellow slurry. Filter the slurry collecting the clear filtrate. Wash the filtrate with aqueous HCl (1 M, 50 mL) 3 times. Then wash the filtrate with saturate aqueous NaHCO₃ (50 mL). Remove the solvent under vacuum to provide an orange oil. Redissolve the orange oil in EtOAc 100 mL. Remove the EtOAc under vacuum if an aqueous layer is visible in the resulting mixture. Repeat as necessary until dissolution of the residue with EtOAc (100 mL, 40° C.) provides a clear homogeneous solution. Filter off any solids if necessary. Seed the warm 40° C. EtOAc solution with seed crystals of CPTP. Cool the mixture with stirring to allow the title compound to crystallize. Additional crystalline CPTP can be recovered from the mother liquor. (Yield: 7 g, 16.85 mmol, 56.8%)

### X-Ray Powder Diffraction

X-ray powder diffraction analysis is performed with a D4 Endeaver diffractometer, equipped with a CuKα source (λ=1.54056 Å) operating at 40 kV and 50 mA. The sample is scanned from 3° to 40° in 2θ, with a step size of 0.009° in 2θ and a scan rate of ≧1.5 sec per step. Sample displacement errors is corrected using the NIST standard SRM675 (standard peak at 8.8° in 2θ). It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g., The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement. In the present case, a peak position variability of ±0.3, preferably 0.2, and more preferably 0. 1 in 2-theta will take into account these potential variations without hindering the unequivocal identification of the indicated crystal form.

Confirmation of a crystal form may be made based on any unique combination of distinguishing peaks (in units of degrees ° 2θ), typically the more prominent peaks. Crystalline CPTP is characterized by an X-ray powder diffraction pattern having distinguishing peaks at a 2θ value of 11.5° and 19.0°. A well known and accepted method for searching crystal forms in the literature is the “Fink” method, see for example, Bigelow, W. and Smith, J. V. (1965). ASTM Spec. Tech. Publ. STP, 372, 54-89. The Fink method uses the four most intense lines for the initial search followed by the next four most intense lines. As used herein the term “intense peaks” refers to peaks observed in the spectrum great than 5%, preferable greater than 10% over the base line. In accord with the Fink method, based on peak intensities as well as 10 peak position, the desired crystalline form of R-2-(4-cyclopropanesulphonyl-phenyl)-N-pyrazin-2-yl-3-(tetrahydropyran-4-yl)-propionamide may be identified by the presence of peaks at 11.5±0.1°, 17.1°±0.1°, 19.0°±0.1°, and 26.6°, ±0.1° in 2θ; when the pattern is obtained from a copper radiation source. The presence of desired R-2-(4-cyclopropanesulphonyl-phenyl)-N-pyrazin-2-yl-3-(tetrahydropyran-4-yl)-propionamide crystalline form may be further verified by additional peaks at 9.4°±0.1, 15.0±0.1°, 16.5°±0.1°, and 20.7°±0.1° in 2θ; when the pattern is obtained from a copper radiation source. A representative example of an X-ray powder diffraction pattern of CPTP that can be obtained using the procedure described above is illustrated in FIG. 1. Table 4 below is a listing of 14 peaks observed in the x-ray powder diffraction analysis described above.

TABLE 4
X-ray powder diffraction (CuKα radiation source,
λ = 1.54056 Å) peaks of crystalline CPTP

| 2θ Angle (± 0.1°) | Intensity (%) |
|---|---|
| 9.4 | 36 |
| 11.5 | 87 |
| 12.5 | 26 |
| 15.0 | 38 |
| 16.5 | 38 |
| 17.1 | 51 |
| 18.4 | 15 |
| 19.0 | 100 |
| 20.7 | 31 |
| 21.9 | 19 |
| 23.4 | 21 |
| 23.8 | 25 |
| 24.8 | 20 |
| 26.6 | 63 |

### Solid State NMR

¹³C Cross polarization/magic angle spinning (CP/MAS) NMR (solid-state NMR or SSNMR) spectra is obtained using a Bruker Avance II 400 MHz NMR spectrometer operating at a carbon frequency of 100.622 MHz and equipped with a Bruker 4 mm double resonance probe (K299552). TOSS sideband suppression is used along with cross polarization employing SPINAL64 decoupling (95.4 Watts) and a RAMP 100 shaped H-nucleus CP pulse. Acquisition parameters are as follows: 90° proton r.f pulse width of 3.0 μs, contact time was 3.0 ms, pulse repetition time of 25 s, MAS frequency of 10 kHz, spectral width of 30 kHz, acquisition time was 34 ms and the number of scans was 2,187. Chemical shifts are referenced to adamantane (δ=29.5 ppm) in a separate experiment. A representative solid state NMR spectrogram for crystalline CPTP is provided in FIG. 3. Representative resonances from the solid state NMR of crystalline CPTP include: chemical shifts of 172.8, 148.1, 144.7, 143.1, 140.2, 138.1, 133.0, 130.7, 128.2, 126.0, 69.3, 68.7, 49.7, 43.5, 35.2, 34.3, 32.1, 31.5, 6.5, and 5.6 ppm.

What is claimed is:

1. R-2-(4-cyclopropanesulphonyl-phenyl)-N-pyrazin-2-yl-3-(tetrahydropyran-4-yl)-propionamide in crystalline form characterized by an X-ray powder diffraction pattern obtained from a CuKα source (λ=1.54056 Å) which comprises peaks at:
a) 11.5° and 19.0°±0.1° in 2θ; or
b) 11.5°, 17.1°, 19.0°, and 26.6°±0.1° in 2θ; or
c) 11.5°, 17.1°, 19.0°, 26.6°, 29.4°, 15.0, 16.5°, and 20.7°±0.1° in 2θ.

2. R-2-(4-cyclopropanesulphonyl-phenyl)-N-pyrazin-2-yl-3-(tetrahydropyran-4-yl)-propionamide in crystalline form characterized by a solid state NMR spectrum which comprises peaks referenced to adamantine (δ=29.5 ppm) at:
a) δ 172.8, 49.7, and 43.5±10.1 ppm; or
b) δ 172.8, 144.7, 140.2, 49.7, 43.5±0.1 ppm; or
c) δ 172.8, 144.7, 140.2, 49.7, 43.5, 6.5, and 5.6±0.1 ppm.

3. R-2-(4-cyclopropanesulphonyl-phenyl)-N-pyrazin-2-yl-3-(tetrahydropyran-4-yl)-propionamide in crystalline form characterized by at least one of the following:
a) an X-ray powder diffraction pattern which comprises intense peaks at 11.5° and 19.0°±0.1° in 2θ obtained from a CuKα source (λ=1.54056 Å); or
b) an onset of melting using differential scanning calorimetry of 156±3° C.; or
c) a solid state NMR spectrum which comprises peaks at δ 172.8, 49.7, and 43.5±10.1 ppm referenced to adamantine (δ=29.5 ppm).

4. R-2-(4-cyclopropanesulphonyl-phenyl)-N-pyrazin-2-yl-3-(tetrahydropyran-4-yl)-propionamide comprising substantially pure crystalline R-2-(4-cyclopropanesulphonyl-phenyl)-N-pyrazin-2-yl-3-(tetrahydropyran-4-yl)-propionamide according to claim 3.

5. R-2-(4-cyclopropanesulphonyl-phenyl)-N-pyrazin-2-yl-3-(tetrahydropyran-4-yl)-propionamide according to claim 4 wherein comprising substantially pure crystalline R-2-(4-cyclopropanesulphonyl-phenyl)-N-pyrazin-2-yl-3-(tetrahydropyran-4-yl)-propionamide comprises gather than about 95% crystalline R-2-(4-cyclopropanesulphonyl-phenyl)-N-pyrazin-2-yl-3-(tetrahydropyran-4-yl)-propionamide.

6. A pharmaceutically composition comprising R-2-(4-cyclopropanesulphonyl-phenyl)-N-pyrazin-2-yl-3-(tetrahydropyran-4-yl)-propionamide according to claim 3 and at least one other anti-diabetic agent or anti-hyperglycemic agent.

7. A pharmaceutical composition comprising R-2-(4-cyclopropanesulphonyl-phenyl)-N-pyrazin-2-yl-3-(tetrahydropyran-4-yl)-propionamide according to claim 3 and at least one of a pharmaceutically acceptable: carrier, excipient, or diluent.

8. A method of preventing or treating hyperglycemia in a mammal in need of treatment comprising administering an effective amount of a crystalline R-2-(4-cyclopropanesulphonyl-phenyl)-N-pyrazin-2-yl-3-(tetrahydropyran-4-yl)-propionamide according to claim 3.

9. A method of treating diabetes in a mammal in need of treatment comprising a step of administering an effective amount of crystalline R-2-(4-cyclopropanesulphonyl-phenyl)-N-pyrazin-2-yl-3-(tetrahydropyran-4-yl)-propionamide according to claim 3.

10. A method according to claim 9 wherein crystalline R-2-(4-cyclopropanesulphonyl-phenyl)-N-pyrazin-2-yl-3-(tetrahydropyran-4-yl)-propionamide is administered in combination with one or more other anti hyperglycemic agents or antidiabetic agents.
